(19) United States
(12) Patent Application Publication
(10) Pub. No.: US 2025/0231176 A1
(43) Pub. Date: Jul. 17, 2025

(54) OLFACTORY RECEPTOR GENE FOR DIAGNOSING SKIN AGING AND USE THEREOF

(71) Applicant: UIF (University Industry Foundation), Yonsei University, Seoul (KR)

(72) Inventors: Taesun PARK, Seoul (KR); Wesuk KANG, Seoul (KR)

(73) Assignee: UIF (University Industry Foundation), Yonsei University, Seoul (KR)

(21) Appl. No.: 18/700,442

(22) PCT Filed: Oct. 12, 2022
(86) PCT No.: PCT/KR2022/015394

§ 371 Date: Jun. 19, 2024

(30) Foreign Application Priority Data
Oct. 12, 2021 (KR) 10-2021-0135027

(51) Int. Cl. G01N 33/50; C12Q 1/6883; G01N 33/543; G01N 33/68

(52) U.S. Cl. CPC G01N 33/5023; C12Q 1/6883; G01N 33/5044; G01N 33/543; G01N 33/6872; C12Q 2600/158; G01N 2333/705

(57) ABSTRACT

The present disclosure relates to a composition for diagnosing skin aging. The composition of the present disclosure can be usefully used to establish a personalized treatment strategy for skin aging early not only by evaluating the current level of skin aging affected by endogenous factors but also by predicting the genetic risk of future skin aging with high reliability. The present disclosure also relates to a method for screening a skin aging alleviating agent. The screening method of the present disclosure allows accurate and fast screening of a fundamental skin aging inhibitor that goes beyond symptomatic therapy such as moisture supply by restoring collagen production in dermal fibroblasts reduced with aging of the skin's dermis to reversibly restore decreased binding strength, loss of elasticity, loss of moisture, and damage to barrier function of skin tissue.

### TECHNICAL FIELD

The present disclosure relates to an olfactory receptor gene which is an indicator of skin dermal aging and a method for diagnosing skin aging using the same.

### BACKGROUND ART

The skin is mainly composed of three layers: the epidermis, dermis and hypodermis. The epidermis, especially the stratum corneum, which is the outermost layer of the epidermis, acts as a skin barrier and suppresses the loss of moisture and electrolytes from the skin, while the dermal layer is composed mainly of connective tissue and plays a role in maintaining the elasticity and structure of the skin. The skin's connective tissue contains the highest amount of collagen among extracellular matrix proteins. It is also composed of proteins such as elastin, fibronectin, integrin, fibrillin and proteoglycans and is filled with hyaluronic acid which is responsible for moisturizing the skin (Fore, J., Ostomy Wound Management, 52 (9): 24, 2006; Yamauchi et al., Journal of Biological Chemistry, 262 (24): 11428, 1987).

Collagen is produced from procollagen that has been newly synthesized in dermal fibroblasts, and after being secreted into the extracellular space of skin cells through an enzymatic reaction, it forms microfibrils with a triple helix configuration. The microfibrils combine with leucine-rich small proteoglycans to form fibrils. The fibrils produced in this way assemble to form collagen fibers that provide skin adhesion and elasticity, and are involved in the mechanical rigidity of the skin, the binding power and elasticity of the tissue, etc. (Veis, A et al., Journal of Biological Chemistry, 264 (7): 3884, 1989; Hulmes, D. J., Journal of Structural Biology, 137 (1): 2, 2002).

As skin aging progresses, the content of collagen, which accounts for most of the skin's connective tissue, decreases. Since collagen imparts tension and strength to the skin, the decrease in collagen due to skin aging has been identified as a direct cause of symptoms such as wrinkles and decreased elasticity. This natural aging phenomenon can be confirmed by the decrease in collagen and increase in cellular aging factors (p16, β-galactosidase, etc.) depending on passages in cell experiments (Yang et al., Experimental Gerontology, 40 (10): 813-819, 2005; Ohtani et al., Nature, 409 (6823): 1067-1070).

Recently, as personalized medicine based on genetic information has emerged as a new paradigm in the health industry, the number of companies that perform diagnostic tests and direct-to-consumer (DTC) tests is increasing gradually, and the genetic testing market is expected to grow at an average annual rate of 10.6% from 5.82 billion dollars (about 7.7 trillion won) in 2017 to 11.79 billion dollars (about 14.3 trillion won) in 2024 (Biotechnology Policy Research Center, 2019). Currently, DTC genetic testing kits in Korea are manufactured by various biocompanies such as Teragen Bio and Amorepacific's “IOPE Lab Gino Index,” Macrogen's “My Genome Story The Plus” and Bioneers' “Gene to Life,” and most of them focus on determining how vulnerable individuals are to related diseases such as hyperglycemia, hypertension, hair loss, hypercholesterolemia, etc. by measuring their innate gene expression status through biological DNA analysis. However, there are no diagnostic kits that reflect the current progress of skin aging rather than the innate genetic information. Also, since the commercially available diagnostic kits collect samples from oral epithelial cells rather than from skin to comprehensively test genes for various items, there is an urgent need for complementary products that can test skin aging conditions directly from the skin.

Meanwhile, it is known that olfactory receptors (ORs) are mainly expressed in olfactory epithelial cells. When odor molecules in the air bind to olfactory receptors present on the cell membranes of olfactory epithelial cells, G_{olf} (olfactory G protein) is activated first and AC3 (olfactory-related adenylate cyclase) is activated immediately. The latter promotes the production of CAMP (cyclic AMP) from ATP within olfactory cells, activates calcium ion channels, and ultimately transmits signals to the brain to recognize the odor molecules (Fleischer et al., Frontiers in Cellular Neuroscience 3:9, 2009). It has been reported that approximately 350 olfactory receptor isoforms are expressed in humans. It is a large gene pool that amounts to 1% or more of the total genomic genes, and is known as the genes corresponding to the largest group of GPCRs. A single olfactory receptor is responsible for about 2-3 odor molecules, and humans can distinguish 10,000 different odors (or odor molecules) via a combination of about 100 olfactory receptors (Pluznick et al., Distinctions of the National Academy of Sciences, 106 (6): 2059-2064, 2009).

According to a recent research, olfactory receptors are expressed widely not only in olfactory tissues, but also in non-olfactory tissues such as skin, lungs, bladder, and large intestine. It is known that the expression varies depending on the condition of the body. For example, the expression of the OR10G7 gene was higher in the skin of atopic patients than in normal human skin (Tham et al., Journal of Allergy and Clinical Immunology, 143 (5): 1838-1848. 2019) and the expression of the OR2AG2 gene in lung tissue of patients with asthma was reported to be lower than that of normal people. They were presented as markers that can predict current disease status and sensitivity (Chakraborty et al., Scientific Reports 9 (1): 1-10, 2019). Also, the OR10H1 gene, the expression of which was significantly higher in the bladder of patients with bladder cancer compared to normal people, was proposed as a biomarker for early diagnosis of bladder cancer (Weber, L et al., Frontiers in Physiology, 9:456, 201 8), and the OR7C1 gene, the expression of which in the large intestine was higher in patients with colorectal cancer than normal people, was presented as a new marker for diagnosing colorectal cancer (Morita et al., Clinical Cancer Research, 22 (13): 3298-3309, 2016). Therefore, although it has been reported in the past few years that ectopic expression of olfactory receptors can act as a marker for diagnosing and representing physiological and pathological conditions beyond simply recognizing the sense of smell, there is no significant knowledge about whether the expression of olfactory receptors in dermal fibroblasts in the dermis changes during skin aging.

Throughout this specification, a number of papers and patent documents are referred to and citations are shown. The disclosures of the cited papers and patent documents are incorporated in this specification as references in their entirety to more clearly explain the level of the technical field to which the present disclosure belongs and the content of the present disclosure.

### REFERENCES OF RELATED ART

Non-patent document 1. Romain Duroux et al., A Rose Extract Protects the Skin against Stress Mediators: A Potential Role of Olfactory Receptors. Molecules 25:4743 (2020).

### DISCLOSURE

### Technical Problem

The inventors of the present disclosure have made consistent efforts to develop a molecular diagnostic method for accurately evaluating the degree of progression of skin aging. As a result, they have found out that the expression of specific olfactory receptor genes changes significantly in the dermal tissue of aging skin and, by measuring their expression levels, it is possible to predict with high reliability not only the current degree of skin aging but also the genetic risk of loss of mechanical rigidity, binding strength, elasticity and barrier function of skin tissue in the future and to monitor the degree of improvement in skin aging during treatment, and have completed the present disclosure.

The present disclosure is directed to providing a composition and a kit for diagnosing skin aging, and a method for diagnosing skin aging using the same.

The present disclosure is also is directed to providing a method for screening a composition for improving skin aging.

Other purposes and advantages of the present disclosure are made clearer by the detailed description, claims and drawings.

### Technical Solution

According to one aspect of the present disclosure, the present disclosure provides a composition for diagnosing skin aging, which contains an agent for measuring the expression level of one or more gene selected from a group consisting of OR1J2 (olfactory receptor, family 1, subfamily J, member 2), OR1J4 (olfactory receptor, family 1, subfamily J, member 4), OR1L3 (olfactory receptor, family 1, subfamily L, member 3), OR1Q1 (olfactory receptor, family 1, subfamily Q, member 1), OR2AG2 (olfactory receptor, family 2, subfamily AG, member 2), OR2C1 (olfactory receptor, family 2, subfamily C, member 1) and OR52N4 (olfactory receptor, family 52, subfamily N, member 4) or a protein encoding the same as an active ingredient.

The inventors of the present disclosure have made consistent efforts to develop a molecular diagnostic method for accurately evaluating the degree of progression of skin aging. As a result, they have found out that the expression of specific olfactory receptor genes (OR1J2, OR1J4, OR1L3, OR1Q1, OR2AG2, OR2C1 and OR52N4) in dermal tissue is increased specifically when skin aging is caused by external or internal factors and, thus, it is possible to provide information on the degree of skin aging and the stage of skin aging progression and, furthermore, to predict the genetic risk of loss of mechanical rigidity, binding strength, elasticity and barrier function of the skin tissue with high reliability, and have completed the present disclosure.

In this specification, the term “diagnosis” includes the determination of an individual's susceptibility to skin aging, determination of the degree of skin aging that has progressed in the individual, and determination of the prognosis of the individual related to future skin aging. As shown in the examples described later, the expression of olfactory receptor genes discovered in the present disclosure is not only closely related to changes in expression of β-gal and p16, which are markers of skin aging, but also closely related to changes in the expression of collagen, which is a marker for the rigidity and elasticity of skin tissue. Therefore, the term “diagnosis of skin aging” may include the meaning of “evaluation of skin barrier function” or “diagnosis of a skin disease related to collagen deficiency.”

In this specification, the term “skin disease related to collagen deficiency” includes all pathological conditions caused by quantitative loss of collagen in the dermis due to decreased collagen production in fibroblasts in cutaneous dermal tissue, increased expression of the collagen gene (COL1A1), or degradation of collagen by MMP-1 (collagenase) faster than collagen synthesis by fibroblasts. Unlike the skin's epidermis, which is densely populated with keratinocytes, most of the skin's dermis consists of cell-free matrix proteins. Collagen is the most important matrix protein that accounts for 75% of the dry weight of skin. The quantitative loss of collagen leads to deficiency of the total matrix protein and structural collapse of dermal tissue, causing decreased binding strength of skin tissue, loss of elasticity, loss of moisture, and damage to barrier function. Accordingly, the composition of the present disclosure can accurately predict the increase or decrease in collagen production through the expression pattern of olfactory receptor genes in fibroblasts, which are the only cellular components in dermal tissue, and can provide important information on the skin damage caused by collagen deficiency and the genetic risk of future damage.

According to a specific embodiment of the present disclosure, the skin aging which can be diagnosed with the composition of the present disclosure may be aging caused by external or internal factors, and the skin aging caused by internal factors may be a natural aging phenomenon that occurs over time, showing wrinkle formation and loss of skin elasticity. Also, the skin aging caused by external factors described above may be caused by factors derived from outside, and may show one or more aging phenomena selected from a group consisting of fine wrinkles, hyperpigmentation, loss of skin elasticity, and moisture deficiency due to skin aging caused by smoking, drinking, lack of nutrition, ultraviolet rays, stress and inflammation.

In this specification, the term “diagnostic composition” refers to an integrated mixture or device containing a means for measuring the expression level of the olfactory receptor genes described above or proteins thereof to determine the degree of skin aging of a subject or predict the progression of future skin aging, and can also be expressed as a “diagnostic kit”.

According to a specific embodiment of the present disclosure, the composition includes an agent for measuring the expression level of one or more gene selected from a group consisting of OR1J2, OR1J4, OR1L3, OR1Q1, OR2AG2, OR2C1 and OR52N4, or a protein encoded thereby. More specifically, the composition of the present disclosure can be used to predict the degree of skin aging when it contains an agent for measuring the expression level of the genes or proteins described above. More specifically, if one or more gene selected from a group consisting of OR1J2, OR1J4, OR1L3, OR1Q1, OR2AG2, OR2C1 and OR52N4 or a protein encoded thereby in a sample of the subject is highly expressed as compared to a normal sample, it is determined that skin aging has occurred due to internal or external factors.

According to a specific exemplary embodiment of the present disclosure, the skin aging due to internal factors is not particularly limited as long as it includes natural aging phenomena that occur over time. It can comprehensively include all skin aging phenomena directly or indirectly caused by this, and may specifically include weakening of one or more skin barrier functions selected from a group composed of fine wrinkles, skin dryness, loss of skin elasticity, reduction of skin thickness, and reduction of collagen, elastin and sebum secretion.

According to a specific embodiment of the present disclosure, the skin aging due external factors described above may be skin aging caused by smoking, drinking, lack of nutrition, exposure to ultraviolet rays, stress, inflammation, etc. For example, the skin aging caused by UV exposure includes one or more photoaging phenomena selected from a group consisting of fine wrinkles, hyperpigmentation, loss of skin elasticity and moisture deficiency. But, without being limited thereto, it comprehensively includes all skin aging phenomena directly or indirectly caused by UV stimulation.

The skin aging caused by stress described above comprehensively includes all skin aging phenomena directly or indirectly caused by stresses caused by physical, psychological and physiological stimuli. In this specification, the term “stress” encompasses all changes within an organism caused by internal and external stimuli that disrupt biological balance. The stimuli that cause stress are broadly classified as physical, psychological and physiological stimuli. The physical stimuli include temperature, ultraviolet rays and chemical stimuli that exist in nature, the psychological stimuli include emotional distress, anger, anxiety, tension, etc., and the physiological stimuli include bacteria, viruses, allergens, etc.

The skin aging caused by inflammation includes skin aging caused by inflammatory or autoimmune skin diseases selected from a group consisting of atopic dermatitis, dermatomyositis, scleroderma, pemphigus, annular erythema and lupus erythematosus. Without being limited thereto, it comprehensively includes all skin aging phenomena caused directly or indirectly by excessive immune response or skin damage caused by inflammation.

According to a specific embodiment of the present disclosure, the composition contains an agent for measuring the expression level of one or more gene selected from a group consisting of OR1J2, OR1J4, OR1L3, OR1Q1, OR2AG2, OR2C1 and OR52N4, or a protein encoded thereby. More specifically, when the composition of the present disclosure contains an agent for measuring the expression of the genes or proteins described above, it may be used to predict the degree of skin aging caused by damage to the barrier function of the skin. More specifically, when one or more of the genes or proteins encoded by them are expressed more highly in the sample of the subject than in the normal sample, it is determined that aging has occurred in the skin of the subject due to damage to barrier function.

In this specification, the term “skin barrier dysfunction-related disease” encompasses a series of diseases caused by the loss of barrier function of the skin that blocks the permeation of harmful molecules from the outside and suppresses loss of internal moisture caused by the loss of collagen and decrease in the thickness of the dermal layer. Loss of barrier function causes various pathological conditions, such as skin aging due to loss of moisture caused by decreased thickness of the dermal layer, various infectious diseases due to the penetration of pathogens, and inflammatory and autoimmune diseases such as atopic dermatitis and contact dermatitis. According to the present disclosure, the inventors of the present disclosure have induced skin aging by increasing the number of passages to cause decrease in skin barrier function caused by internal factors and, as a result, they have confirmed that the expression of aging markers has increased in dermal fibroblasts, the synthesis of collagen has decreased and, at the same time, the expression of the olfactory receptor genes described above has increased significantly.

In this specification, the term “high expression” refers to a case where the expression level of the olfactory receptor gene of the present disclosure or the protein encoded thereby, which has been identified as a positive marker for skin aging, is significantly higher than in an individual wherein aging has not progressed. Specifically, it means a case where the expression level is 130% or higher, more specifically 150% or higher, most specifically 170% or higher, as compared to a normal individual, although not being limited thereto.

According to a specific embodiment of the present disclosure, the agent for measuring the expression level of the gene of the present disclosure is a primer or a probe that specifically binds to the nucleic acid molecule of the gene.

In this specification, the term “nucleic acid molecule” encompasses DNA (gDNA and cDNA) and RNA molecules, and nucleotides, which are basic structural units in nucleic acid molecules, include not only natural nucleotides, but also analogues with modified sugar or base moieties (Scheit, Nucleotide Analogs, John Wiley, New York (1980); Uhlman and Peyman, Chemical Reviews, 90:543-584 (1990)).

The term “primer” used in this specification refers to an oligonucleotide that acts as a starting point for synthesis under the condition where the synthesis of a primer extension product complementary to a nucleic acid chain (template) is induced, i.e., at a suitable temperature and pH in the presence of nucleotides and a polymerase such as a DNA polymerase. Specifically, the primer is a single-chain deoxyribonucleotide. The primer used in the present disclosure may include naturally occurring dNMPs (i.e., dAMP, dGMP, dCMP and dTMP), modified nucleotides, or non-natural nucleotides, and can also include ribonucleotides.

The primer of the present disclosure can be an extension primer that is annealed to a target nucleic acid to form a sequence complementary to the target nucleic acid by a template-dependent nucleic acid polymerase, which extends to the annealed position of the immobilized probe and occupies the annealed area of the probe.

The extension primer used in the present disclosure includes a hybridized nucleotide sequence complementary to a target nucleic acid, e.g., a specific nucleotide sequence of the olfactory receptor gene described above. The term “complementary” means that a primer or a probe is sufficiently complementary to selectively hybridize to a target nucleic acid sequence under predetermined annealing or hybridization conditions, and encompasses both cases where it is substantially complementary and perfectly complementary. More specifically, it means a cases where it is perfectly complementary. The term “substantially complementary sequence” in this specification includes not only a sequence that matches completely but also a sequence that partially matches the compared sequence, to the extent that it can act as a primer by annealing to the specific sequence.

The primer should be long enough to prime the synthesis of extension products in the presence of a polymerase. The appropriate length of the primer is determined by a number of factors, such as temperature, pH, and the source of the primer, but is typically 15-30 nucleotides. Short primer molecules generally require a lower temperature to form a sufficiently stable hybrid complex with the template. The design of such primers can be easily carried out by those skilled in the art by referring to the target nucleotide sequence using, for example, a program for primer design (e.g., PRIMER 3).

In this specification, the term “probe” refers to a linear oligomer having natural or modified monomers or bonds, including deoxyribonucleotides and ribonucleotides that can be hybridized to a specific nucleotide sequence. Specifically, the probe is single-stranded for maximum efficiency in hybridization, and more specifically, it is a deoxyribonucleotide. As the probe used in the present disclosure, a sequence that is perfectly complementary to a specific nucleotide sequence of each olfactory receptor gene described above can be used, but a substantially complementary sequence can also be used as long as it does not interfere with specific hybridization. Generally, since the stability of a duplex formed by hybridization tends to be determined by the matching of terminal sequences, it is preferable to use a probe complementary to the 3′- or 5′-end of the target sequence. Conditions suitable for hybridization can be determined by referring to the matters described in Joseph Sambrook, et al., Molecular Cloning, A Laboratory Manual, Cold Spring Harbor Laboratory Press, N.Y. (2001) and Haymes, B. D., et al., Nucleic Acid Hybridization, A Practical Approach, IRL Press, Washington, D.C. (1985).

According to a specific embodiment of the present disclosure, the agent for measuring the expression level of the protein of the present disclosure may be an antibody that specifically binds to the protein or an antigen-binding fragment thereof, or an aptamer that specifically binds to the protein.

According to the present disclosure, the olfactory receptor protein of the present disclosure can be detected by an immunoassay method using an antigen-antibody reaction and can be used to analyze whether an individual has lost epithelial barrier function. This immunoassay can be performed according to various immunoassay or immunostaining protocols previously developed.

For example, when the method of the present disclosure is performed by a radioimmunoassay method, antibodies labeled with radioactive isotopes (e.g., C¹⁴, I¹²⁵, P³² and S³⁵) can be used.

In the present specification, the term “antibody” refers to an immunoglobulin protein containing one or more variable domains that bind to an epitope of an antigen and specifically recognizing that antigen, produced by the mammalian immune system. The antibody that specifically recognizes each olfactory receptor protein in the present disclosure is a polyclonal or monoclonal antibody, specifically a monoclonal antibody.

The antibodies of the present disclosure can be prepared by methods commonly implemented in the art, such as fusion methods (Kohler and Milstein, European Journal of Immunology, 6:511-519 (1976)), recombinant DNA methods (U.S. Pat. No. 4,816,567), or phage antibody library methods (Clackson, Nature, 352:624-628 (1991)) and Marks et al., J. Mol. Biol., 222:58, 1-597 (1991)). The general processes for preparing antibodies are described in detail in Harlow, E. and Lane, D., Using Antibodies: A Laboratory Manual, Cold Spring Harbor Press, New York, 1999; and Zola, H., Monoclonal Antibodies: A Manual of Techniques, CRC Press, Inc., Boca Raton, Florida, 1984.

Epithelial barrier function can be predicted by analyzing the intensity of the final signal according to the immunoassay process described above. In other words, if a signal for each olfactory receptor, which is a positive marker, is detected more strongly, or if an olfactory receptor, which is a negative marker, is detected more weakly as compared to a normal sample, it is determined that the skin barrier function of the individual is lost or decreased and skin aging has progressed.

In this specification, the term “antigen-binding fragment” refers to a region of a polypeptide to which an antigen can bind in the overall structure of an immunoglobulin, and includes, for example, F(ab′)₂, Fab′, Fab, Fᵥ and scFᵥ, although not being limited to thereto.

In this specification, the term “specific binding” has the same meaning as “specific recognition” and means that an antigen and an antibody (or a fragment thereof) interact specifically through an immunological response.

In the present disclosure, an aptamer which specifically binds to each olfactory receptor protein can also be used instead of the antibody. In this specification, the term “aptamer” refers to a single-stranded nucleic acid (RNA or DNA) molecule or a peptide molecule that binds to a specific target substance with high affinity and specificity. General contents about aptamers are described in detail in Hoppe-Seyler F, Butz K “Peptide aptamers: Powerful New Tools for Molecular Medicine.” J Mol Med. 78 (8): 426-30 (2000) and Cohen B A, Colas P, Brent R. “An artificial cell-cycle inhibitor isolated from a combinatorial library”. Proc Natl Acad Sci USA. 95 (24): 14272-7 (1998).

According to another aspect of the present disclosure, the present disclosure provides a skin aging diagnostic kit containing a composition for diagnosing skin aging, which contains an agent for measuring the expression level of one or more gene selected from a group consisting of OR1J2 (olfactory receptor, family 1, subfamily J, member 2), OR1J4 (olfactory receptor, family 1, subfamily J, member 4), OR1L3 (olfactory receptor, family 1, subfamily L, member 3), OR1Q1 (olfactory receptor, family 1, subfamily Q, member 1), OR2AG2 (olfactory receptor, family 2, subfamily AG, member 2), OR2C1 (olfactory receptor, family 52, subfamily N, member 4) and OR52N4 (olfactory receptor, family 52, subfamily N, Member 4), or a protein encoded thereby as an active ingredient.

The kit may be an RT-PCR (reverse polymerase chain reaction) kit, a DNA chip kit, an ELISA (enzyme-linked immunosorbent assay) kit, a protein chip kit, a rapid kit, or an MRM (multiple reaction monitoring) kit.

The kit of the present disclosure may include, in addition to the agent for measuring the expression level of one or more gene selected from a group consisting of OR1J2, OR1J4, OR1L3, OR1Q1, OR2AG2, OR2C1, and OR52N4 or a protein encoded thereby, one or more different compositions, solutions or devices suitable for analytical methods, although they do not limit the scope of the present disclosure in any way.

In addition to a primer pair specific for the marker gene, the RT-PCR kit can include a test tube or another suitable container, a reaction buffer, deoxynucleotides (dNTPs), an enzyme such as Taq polymerase and reverse transcriptase, a DNase or RNase inhibitor, DEPC-water, sterile water, etc.

The kit of the present disclosure may be a kit containing essential elements required to perform a microarray chip. The microarray chip kit may include a substrate with cDNA corresponding to a gene or fragment thereof attached as a probe, and the substrate may include a quantitative control gene or a cDNA corresponding to the fragment thereof. It can be prepared easily by a method commonly used in the art using a marker of the present disclosure. In order to manufacture a microarray chip, it is preferable to use a micropipetting method using a piezoelectric method, a method using a pin-shaped spotter, etc. to fix the marker on the substrate of a DNA chip using a probe DNA molecule, although not being limited to thereto. Specifically, the substrate of the microarray chip may be coated with an active group selected from a group consisting of amino-silane, poly-L-lysine and aldehyde. Also, the substrate may be specifically selected from a group consisting of a slide glass, a plastic, a metal, silicon, a nylon film and a nitrocellulose membrane, although not being limited to thereto.

The kit containing the agent for measuring the expression of the protein described above may include a substrate, an appropriate buffer solution, a secondary antibody labeled with a chromogenic enzyme or a fluorescent material, a chromogenic substrate, etc. for immunological detection of antibodies. As the substrate, a nitrocellulose membrane, a 96-well plate synthesized from polyvinyl resin, a 96-well plate synthesized from polystyrene resin, a slide glass made from glass, etc. can be used. As the chromogenic enzyme, peroxidase, alkaline phosphatase, etc. can be used. As the fluorescent material, FITC, RITC, etc. can be used. As the chromogenic substrate, ABTS (2,2′-azino-bis-(3-ethylbenzothiazoline-6-sulfonic acid)), OPD (o-phenylenediamine) or TMB (tetramethylbenzidine) can be used, although not being limited to thereto.

According to another aspect of the present disclosure, the present disclosure provides a method for providing information necessary for diagnosing skin aging, which includes a step of measuring the expression level of one or more gene selected from a group consisting of OR1J2, OR1J4, OR1L3, OR1Q1, OR2AG2, OR2C1 and OR52N4 or a protein encoded thereby in a biological sample isolated from an individual.

The description of the olfactory receptor genes, which are novel biomarkers discovered in the present disclosure, and diagnosis of skin aging using the same will be omitted to avoid redundancy since they have already been described above.

The inventors of the present disclosure have identified the correlation between the olfactory receptor genes described above and skin aging for the first time. Thus, it is possible to evaluate the degree of skin aging of an individual using the expression level of the olfactory receptor genes or the proteins encoded thereby as markers.

In this specification, the term “individual” refers to an individual who provides a sample for measuring the expression of each olfactory receptor gene or its protein, and is ultimately the subject of analysis for skin aging. The individual includes, without limitation, human, mouse, rat, guinea pig, dog, cat, horse, cow, pig, monkey, chimpanzee, baboon or rhesus monkey. Specifically, the individual is human. Since the composition of the present disclosure provides information to predict not only the current degree of skin aging but also the genetic risk that the skin aging process will be accelerated in the future, the “individual” of the present disclosure may be a patient whose skin aging has progressed or skin barrier function has decreased, or it may be a normal (healthy) subject whose skin aging has not progressed yet.

In this specification, the term “biological sample” refers to any sample obtained from a mammal, including human, containing cells expressing the olfactory receptor genes described above, and includes, but is not limited to, tissues, organs, cells or cell cultures. Specifically, the biological sample contains skin tissues or cells derived from skin tissues, and more specifically includes cutaneous dermal tissues or ells derived from cutaneous dermal tissues, and most specifically includes dermal fibroblasts.

According to another aspect of the present disclosure, the present disclosure provides a method for screening a composition for improving skin aging, which includes:
(a) a step of contacting a candidate substance with a biological sample containing cells expressing one or more gene selected from a group consisting of OR1J2, OR1J4, OR1L3, OR1Q1, OR2AG2, OR2C1 and OR52N4; and
(b) a step for measuring the expression level or activity of the gene or a protein encoded by the gene in the sample,
wherein, when the expression level or activity of one or more gene selected from a group consisting of OR1J2, OR1J4, OR1L3, OR1Q1, OR2AG2, OR2C1 and OR52N4 or a protein encoded thereby increases, the candidate substance is determined to be a composition for improving skin aging.

According to a specific embodiment of the present disclosure, the cells used in the screening method of the present disclosure are skin tissue-derived cells, more specifically cutaneous epidermal tissue-derived cells, and most specifically dermal fibroblasts.

The term “candidate substance” used when referring to the screening method of the present disclosure refers to an unknown substance used in screening to test whether it affects the activity or expression level of each olfactory receptor gene or protein by being added to the sample containing cells expressing the olfactory receptors described above. The candidate substance includes, but is not limited to, a compound, a nucleotide, a peptide and a natural extract. The step of measuring the expression level or activity of each olfactory receptor in the biological sample treated with the test substance can be performed by various expression and activity measurement methods known in the art.

In this specification, the term “increase in activity or expression” means an increase in the unique function or expression level of olfactory receptors, which are positive markers for skin aging, in vivo to the extent that endogenous skin aging and damage to skin barrier function caused by internal or external factors are improved to a measurable level, specifically, increase in the activity or expression level by 130%, more specifically by 150% or more, most specifically by 170% or more, as compared to a control group that has not been treated with the candidate substance, although not being limited thereto.

### Advantageous Effects

The features and advantages of the present disclosure are summarized as follows:
(a) A composition of the present disclosure can be usefully used to establish a personalized treatment strategy for skin aging early not only by evaluating the current level of skin aging caused by various physiological and external factors but also by predicting the genetic risk of future skin aging with high reliability.
(b) A method for screening a composition for improving skin aging of the present disclosure can accurately and quickly search for underlying skin aging inhibitors that go beyond symptomatic therapy such as hydration by restoring collagen production in dermal fibroblasts reduced by aging of the skin's dermis to reversibly restore decreased binding strength, loss of elasticity, loss of moisture, and damage to barrier function in skin tissue.

### BRIEF DESCRIPTION OF DRAWINGS

FIG. 1 shows a result of measuring change in the expression of β-galactosidase in dermal fibroblasts depending on passages. FIG. 1A shows unaged normal dermal fibroblasts (Young), and FIG. 1B shows aged dermal fibroblasts (Old).

FIG. 2 shows a result of quantifying change in the expression of β-galactosidase in dermal fibroblasts depending on passages. Each value is shown as average±standard error (SEM), and significant difference between groups is displayed by an asterisk (* p<0.05).

FIG. 3 shows a result of quantifying change in the expression of the p16 gene in dermal fibroblasts depending on passages. Each value is shown as average±standard error (SEM), and significant difference between groups is displayed by an asterisk (* p<0.05).

FIG. 4 shows a result of measuring collagen content in dermal fibroblasts depending on passages. Each value is shown as average±standard error (SEM), and significant difference between groups is displayed by an asterisk (* p<0.05).

FIG. 5 shows change in the expression of COL1A1 in dermal fibroblasts depending on passages. Each value is shown as average±standard error (SEM), and significant difference between groups is displayed by an asterisk (* p<0.05).

FIG. 6 shows change in the expression of MMP1 in dermal fibroblasts depending on passages. Each value is shown as average±standard error (SEM), and significant difference between groups is displayed by an asterisk (* p<0.05).

FIG. 7 shows change in the expression of olfactory receptor genes in dermal fibroblasts depending on passages. Each value is shown as average±standard error (SEM), and significant difference between groups is displayed by an asterisk (* p<0.05).

### MODE FOR INVENTION

Hereafter, the present disclosure will be described in more detail through examples. These examples are intended solely to explain the present disclosure in more detail, and it will be obvious to those having common knowledge in the art that the scope of the present disclosure is not limited by the examples.

### EXAMPLES

### Example 1: Construction of Skin Aging Model by Increasing Passages

### 1) Culturing of Dermal Fibroblasts and Construction of Aging Model

Dermal fibroblasts (Hs68) purchased from ATCC (Manassas, VA, USA) were cultured under the condition of 37° C. and 5% CO₂ using DMEM (Dulbecco's modified Eagle's medium; Hyclone, UT, USA) supplemented with 10% fetal bovine serum (HyClone, Logan, UT, USA) and 1% antibiotics (penicillin and streptomycin; GIBCO). Subculturing was carried out after separating the cells from a plate using a trypsin-ethylenediaminetetraacetic acid (EDTA) solution for 5 minutes at 37° C. The solution containing the cells was then transferred to a 15-mL tube and, after centrifuging the cells at 250×g for 5 minutes, the upper layer was removed and the lower layer of cells was pipetted gently into a culture medium and then dispensed back to the plate. The cells at passage 5 were used as unaged normal dermal fibroblasts (Young), and the cells at passage 30 were used as a model of aged dermal fibroblasts (Old).

### 2) β-Galactopyranoside Assay (SA-β-Gal)

To confirm whether aging is induced in the dermal fibroblasts depending on passages, the unaged cells (young) and old the cells (old) were seeded onto a plate, and the expression of β-galactosidase was confirmed using a senescence β-galactosidase synthesis kit (Cell Signaling Technology Co.).

### 3) Collagen Content

In order to compare collagen metabolic activity in dermal fibroblasts, which is related to elasticity and wrinkles, depending on passages, the unaged cells (Young) and the aged cells (Old) were inoculated into a 24-well plate at a concentration of 5×10⁴ cells/well and then cultured for 24 hours. A serum-free culture was added after washing the cells twice with phosphate-buffered saline (PBS). 24 hours later, the cells were treated with a sample and cultured for 48 hours. The culture was collected and used for measurements. The degree of collagen synthesis in the cell culture was determined by measuring collagen content using a procollagen type I C-peptide assay kit (Takara Bio Inc.).

### 4) RNA Extraction and Real-Time PCR

Real-time PCR was performed to quantitatively analyze the genes related to skin aging that change depending on passages in the dermal fibroblasts. To extract RNA from the cells, the culture was removed and washed with cold PBS. Then, total RNA was separated using a TRIzol reagent (Invitrogen, Carlsbad, CA, USA) and the RNA was quantified with a spectrophotometer (NanoQuant Infinite M200 Pro; Tecan, Mannedorf, Switzerland). A reverse transcriptase (SuperScript IV reverse transcriptase; Invitrogen, Carlsbad, CA, USA) was used to synthesize cDNA from the extracted RNA.

Real-time PCR was performed by adding a primer and the cDNA to a SYBR green supermix (BioRad, Hercules, CA, USA) and repeating 36 cycles of 10 seconds at 95° C., 15 seconds at 58° C., and 15 seconds at 60° C. using a CFX Connect™ real-time PCR detection system (BioRad), and the result was confirmed with Bio-Rad CFX Manager 3.0. The expression of each gene was compared and analyzed by normalizing to the expression of GAPDH (glyceraldehyde 3-phosphate dehydrogenase). Primers specific to the genes to be detected were prepared. The nucleotide sequences of the primers used are shown in Table 1.

TABLE 1

| Genes | Primer sequence (5′ → 3′) | |
|---|---|---|
| COL1A1 | F | ACATGTTCAGCTTTGGACC (SEQ ID NO 15) |
| COL1A1 | R | TGTACGCAGGTGATTGGG (SEQ ID NO 16) |
| MMP1 | F | AGGGGAGATCATCGGACAACTC (SEQ ID NO 17) |
| MMP1 | R | GAGAGTCCAAGAATGCCGA (SEQ ID NO 18) |
| p16 | F | TGCCCAACGCAACCGAATAG (SEQ ID NO 19) |
| p16 | R | CCCCTGCAAACTTCGTCCTC (SEQ ID NO 20) |
| GAPDH | F | AAATCAAGTGGGGGATGC (SEQ ID NO 21) |
| GAPDH | R | AGGGGCAGAGATGATGACC (SEQ ID NO 22) |

### 5) Statistical Analysis

All experimental results were presented as mean±standard error, and significance was verified at p<0.05 level by performing an independent samples t-test (Student's t-test) using SPSS Statistics (version 24.0, Chicago, IL, USA).

### Test Results

### 1) Confirmation of Skin Aging Depending on Passages

β-Gal and p16 are typical aging markers for identifying cellular senescence. β-Galactosidase, represented by β-gal, is an enzyme that hydrolyzes breaks down glucose and galactose by hydrolyzing the glycosidic bond of lactose. p16 is involved in the cell growth cycle, and it is known that its expression increases with cellular aging.

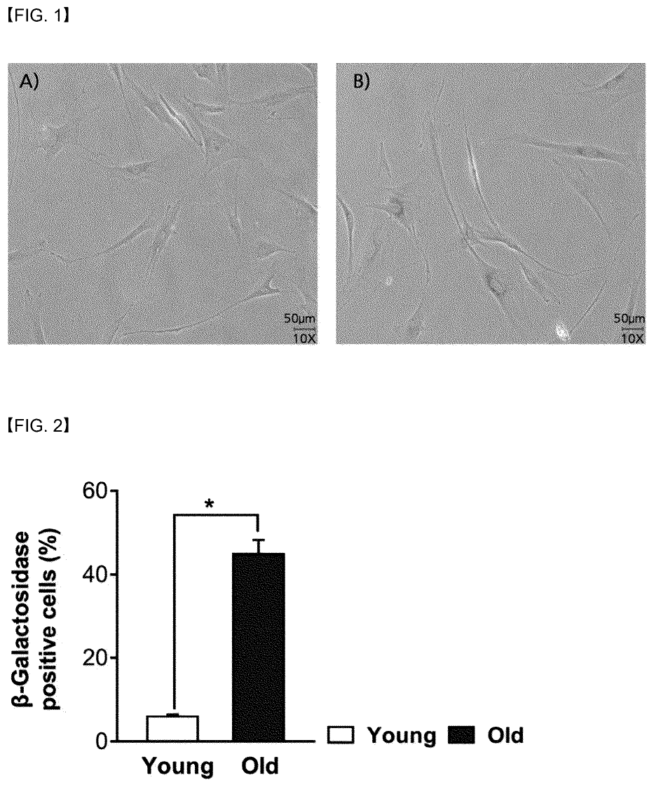

The change in the expression of the aging markers (β-gal and p16) in dermal fibroblasts depending on passages was measured and shown in FIGS. 1 to 3. As shown in FIG. 1 and FIG. 2, it can be seen that beta-galactosidase (β-galactosidase) was significantly overexpressed by 4 to 8 times in the aged human dermal fibroblasts (Old) as compared to the unaged human dermal fibroblasts (Young).

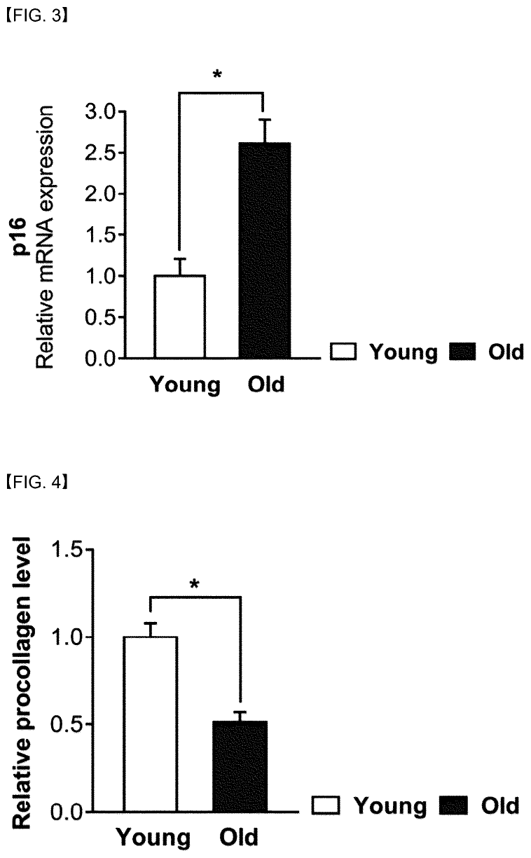

As shown in FIG. 3, it can be seen that the expression of the p16 gene was significantly higher in the aged human dermal fibroblasts (Old) as compared to the unaged human dermal fibroblasts (Young).

In other words, since the expression of the aging markers (β-gal and p16) increased significantly in the aged dermal fibroblasts (Old), it was verified through the experiments of the present disclosure that skin aging can be induced by passages without treatment of external factors.

### 2) Confirmation of Decrease in Collagen Gene and Increase in Collagen-Degrading Gene by Skin Aging

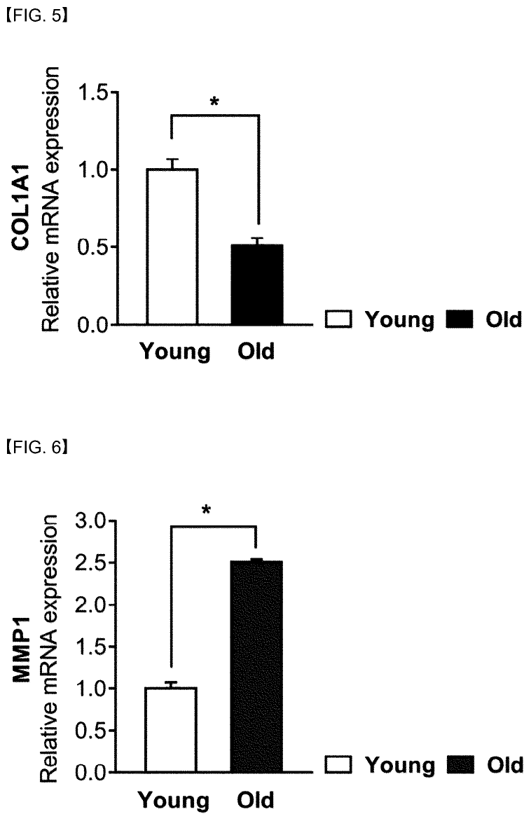

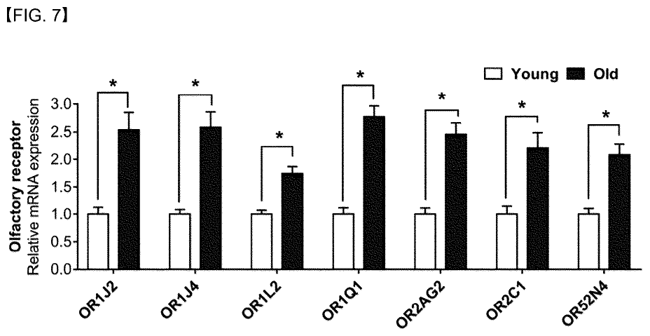

FIGS. 4 to 6 show the result of measuring the production of collagen, the expression of a collagen gene (COL1A1) and the expression of a collagen-degrading enzyme (MMP1) in dermal fibroblasts. It can be seen that the aged dermal fibroblasts (Old) showed significantly lower production of collagen and expression of the collagen gene (collagen, type I, alpha 1; COL1A1) as compared to the unaged dermal fibroblasts (Young).

In contrast, the expression of the collagen-degrading gene (matrix metallopeptidase 1; MMP1) increased significantly in the aged dermal fibroblasts (Old) compared to the unaged dermal fibroblasts (Young).

### Example 2: Change in Expression of Olfactory Receptor Genes Due to Skin Aging

### 1) Culturing of Dermal Fibroblasts and Construction of Aging Model

Human dermal fibroblasts (Hs68) were cultured using the same method as in Example 1, and the models of unaged normal dermal fibroblasts (Young) and aged dermal fibroblasts (Old) were also prepared using the same method as in Example 1.

### 2) RNA Extraction and Real-Time PCR

Real-time PCR was performed to quantitatively analyze the expression pattern of olfactory receptor gene in the models of the unaged normal dermal fibroblasts (Young) and the aged dermal fibroblasts (Old). RNA extraction and real-time PCR were performed using the same methods as in Example 1. Primers specific to the genes to be detected was prepared. The nucleotide sequences of the primers used in Example 2 are shown in Table 2.

TABLE 2

| Genes | Primer sequence (5′ → 3′) | |
|---|---|---|
| OR1J2 | F | TGTCACCCCTCTCCACTAAC (SEQ ID NO 23) |
| OR1J2 | R | CTGGCACAAGACAGAATCCAAG (SEQ ID NO 24) |
| OR1J4 | F | GTCACATCCTCAAGGCTCCA (SEQ ID NO 25) |
| OR1J4 | R | ACCGTGTACATCACAGAGGCA (SEQ ID NO 26) |
| OR1L3 | F | TTCTCTTGGGACTCTCCTCTC (SEQ ID NO 27) |
| OR1L3 | R | CACGAGCATCTTTGGACTAG (SEQ ID NO 28) |
| OR1Q1 | F | TTCTCTCTCTGCTCCACGCCTC (SEQ ID NO 29) |
| OR1Q1 | R | TTTCCATGACGGCGAAGCAA (SEQ ID NO 30) |
| OR2AG2 | F | TCTACAGCCTGGAATAAGGA (SEQ ID NO 31) |
| OR2AG2 | R | GGAGGAATGGGAGGCAA (SEQ ID NO 32) |
| OR2C1 | F | TCAATACGTGCCTCTCCCATC (SEQ ID NO 33) |
| OR2C1 | R | TTCTTGGCCGGAAGCAGATAC (SEQ ID NO 34) |
| OR52N4 | F | CCAAGCTCCTGCCCTACTGC (SEQ ID NO 35) |
| OR52N4 | R | AAGGCCTTCTGCCGAGCATC (SEQ ID NO 36) |
| GAPDH | F | AAATCAAGTGGGGGATGC (SEQ ID NO 37) |
| GAPDH | R | AGGGGCAGAGATGATGACC (SEQ ID NO 38) |

### 3) Statistical Analysis

All experimental results were presented as mean±standard error, and significance was verified at p<0.05 level by performing an independent samples t-test (Student's t-test) using SPSS Statistics (version 24.0, Chicago, IL, USA).

### Test Results

FIG. 7 shows the change in the expression of the olfactory receptor genes in dermal fibroblasts depending on passages. It can be seen that the expression of the olfactory receptor genes OR1J2, OR1J4, OR1L3, OR1Q1, OR2AG2, OR2C1 and OR52N4 in the aged dermal fibroblasts (Old) was significantly increased as compared to the unaged dermal fibroblasts (Young).

TABLE 3
Sequence information of tested olfactory
receptor genes and proteins

SEQ ID NO and Genbank access number

| Olfactory | Amino acid | Nucleotide |
|---|---|---|
| receptors | sequence | sequence |
| OR1J2 | SEQ ID NO 1 | SEQ ID NO 2 |
| | (NP_473448.1) | (NM_054107.1) |
| OR1J4 | SEQ ID NO 3 | SEQ ID NO 4 |
| | (NP_001004452.1) | (NM_001004452.1) |
| OR1L3 | SEQ ID NO 5 | SEQ ID NO 6 |
| | (NP_001005234.1) | (NM_001005234.1) |
| OR1Q1 | SEQ ID NO 7 | SEQ ID NO 8 |
| | (NP_036496.1) | (NM_012364.1) |
| OR2AG2 | SEQ ID NO 9 | SEQ ID NO 10 |
| | (NP_001004490.1) | (NM_001004490.2) |
| OR2C1 | SEQ ID NO 11 | SEQ ID NO 12 |
| | (NP_036500.2) | (NM_012368.3) |
| OR52N4 | SEQ ID NO 13 | SEQ ID NO 14 |
| | (NP_001005175.3) | (NM_001005175.5) |

Although specific exemplary embodiments of the present disclosure have been described in detail above, it is obvious for those having knowledge in the art that they are merely specific exemplary embodiments and, therefore, the scope of the present disclosure is not limited by them. Accordingly, the substantial scope of the present disclosure should be defined by the attached claims and their equivalents.

We claim:

1. A composition for diagnosing skin aging, comprising an agent for measuring the expression level of one or more gene selected from a group consisting of OR1J2 (olfactory receptor, family 1, subfamily J, member 2), OR1J4 (olfactory receptor, family 1, subfamily J, member 4), OR1L3 (olfactory receptor, family 1, subfamily L, member 3), OR1Q1 (olfactory receptor, family 1, subfamily Q, member 1), OR2AG2 (olfactory receptor, family 2, subfamily AG, member 2), OR2C1 (olfactory receptor, family 2, subfamily C, member 1) and OR52N4 (olfactory receptor, family 52, subfamily N, member 4) or protein encoded thereby as an active ingredient.

2. The composition according to claim 1, wherein the skin aging comprises one or more aging phenomena selected from a group consisting of fine wrinkles, hyperpigmentation, loss of skin elasticity and moisture deficiency, caused by internal or external factors.

3. The composition according to claim 2, wherein the composition is a composition for predicting the degree of skin aging caused by internal or external factors.

4. The composition according to claim 1, wherein the agent for measuring the expression of the gene is a primer or a probe that specifically binds to the nucleic acid molecule of the gene.

5. The composition according to claim 1, wherein the agent for measuring the amount of expression of the protein is an antibody that specifically binds to the protein or an antigen-binding fragment thereof, or an aptamer that specifically binds to the protein.

6. A kit for diagnosing skin aging, comprising the composition for diagnosing skin aging according claim 1.

7. The kit according to claim 6, wherein the kit is an RT-PCR (reverse polymerase chain reaction) kit, a DNA chip kit, an ELISA (enzyme-linked immunosorbent assay) kit, a protein chip kit, a rapid kit or an MRM (multiple reaction monitoring) kit.

8. A method for providing information necessary for diagnosing skin aging, comprising a step of measuring the expression level of one or more gene selected from a group consisting of OR1J2, OR1J4, OR1L3, OR1Q1, OR2AG2, OR2C1 and OR52N4 or a protein encoded thereby in a biological sample isolated from an individual.

9. The method according to claim 8, wherein the biological sample comprises skin tissues or skin tissue-derived cells.

10. The method according to claim 9, wherein the skin tissue-derived cells are dermal fibroblasts.

11. A method for screening a composition for improving skin aging, comprising:
(a) a step of contacting a candidate substance with a biological sample comprising cells expressing one or more gene selected from a group consisting of OR1J2, OR1J4, OR1L3, OR1Q1, OR2AG2, OR2C1 and OR52N4; and
(b) a step for measuring the expression level or activity of the gene or a protein encoded by the gene in the sample,
wherein, when the expression level or activity of one or more gene selected from a group consisting of OR1J2, OR1J4, OR1L3, OR1Q1, OR2AG2, OR2C1 and OR52N4 or a protein encoded by the gene increases, the candidate substance is determined to be a composition for improving skin aging.

12. The method according to claim 11, wherein the cells are dermal fibroblasts.


SEQUENCE LISTING (38 sequences)

SEQ ID NO: 1 - protein, 313 aa, Homo sapiens
MSPENQSSVSEFLLLGLPIRPEQQAVFFTLFLGMYLTTVLGNLLIMLLIQLDSHLHTPMYFFLSHLALTDISFSSVTVPKMLMDMRTKYKSILYEECISQMYFFIFFTDLDSFLITSMAYDRYVAICHPLHYTVIMREELCVFLVAVSWILSCASSLSHTLLLTRLSFCAANTIPHVFCDLAALLKLSCSDIFLNELVMFTVGVVVITLPFMCILVSYGYIGATILRVPSTKGIHKALSTCGSHLSVVSLYYGSIFGQYLFPTVSSSIDKDVIVALMYTVVTPMLNPFIYSLRNRDMKEALGKLFSRATFFSW

SEQ ID NO: 2 - DNA, 939 bp, Homo sapiens
  source 1..939: mol_type = genomic DNA
atgagccctgagaaccagagcagcgtgtccgagttcctccttctgggcctccccatccggccagagcagcaggctgtgttcttcaccctgttcctgggcatgtacctgaccacggtgctggggaacctgctcatcatgctgctcatccagctggactctcaccttcacacccccatgtacttcttcctcagccacttggctctcactgacatctccttttcatctgtcactgtccctaagatgctgatggacatgcggactaagtacaaatcgatcctctatgaggaatgcatttctcagatgtatttttttatattttttactgacctggacagcttccttattacatcaatggcatatgaccgatatgttgccatatgtcaccctctccactacactgtcatcatgagggaagagctctgtgtcttcttggctgtatcttggattctgtcttgtgccagctccctctctcacacccttctcctgacccggctgtctttctgtgctgcgaacaccatcccccatgtcttctgtgaccttgctgccctgctcaagctgtcctgctcagatatcttcctcaatgagctggtcatgttcacagtaggggtggtggtcattaccctgccattcatgtgtatcctggtatcatatggctacattggggccaccatcctgagggtcccttcaaccaaagggatccacaaagcattgtccacatgtggctcccatctctctgtggtgtctctctattatgggtcaatatttggccagtaccttttcccgactgtaagcagttctattgacaaggatgtcattgtggctctcatgtacacggtggtcacacccatgttgaacccctttatctacagccttaggaacagggacatgaaagaggcccttgggaaactcttcagtagagcaacatttttctcttggtga

SEQ ID NO: 3 - protein, 313 aa, Homo sapiens
MKRENQSSVSEFLLLDLPIWPEQQAVFFTLFLGMYLITVLGNLLIILLIRLDSHLHTPMFFFLSHLALTDISLSSVTVPKMLLSMQTQDQSILYAGCVTQMYFFIFFTDLDNFLLTSMAYDRYVAICHPLRYTTIMKEGLCNLLVTVSWILSCTNALSHTLLLAQLSFCADNTIPHFFCDLVALLKLSCSDISLNELVIFTVGQAVITLPLICILISYGHIGVTILKAPSTKGIFKALSTCGSHLSVVSLYYGTIIGLYFLPSSSASSDKDVIASVMYTVITPLLNPFIYSLRNRDIKGALERLFNRATVLSQ

SEQ ID NO: 4 - DNA, 939 bp, Homo sapiens
  source 1..939: mol_type = genomic DNA
atgaagagggagaatcagagcagtgtgtctgagttcctcctcctggacctccccatctggccagagcagcaggctgtgttcttcaccctgttcttgggcatgtacctgatcacggtgctggggaacctgctcatcatcctgctcatccggctggactctcaccttcacacccccatgttcttcttcctcagccacttggctctcactgacatctccctttcatctgtcactgtcccaaagatgttattaagcatgcaaactcaggatcaatccattctttatgcagggtgtgtaactcagatgtattttttcatatttttcactgatctagacaatttccttctcacttcaatggcatacgatcggtatgtggccatctgtcaccccctccgctacaccactatcatgaaagagggactgtgtaacttactcactgtgtcctggatcctctcctgtaccaatgccctgtctcacactctcctcctggcccagctgtccttttgtgctgacaacaccatcccccatttcttctgtgatcttgttgccctactcaagctctcatgctcagacatctccctcaatgagctggtcattttcacagtgggacaggcagtcattactctaccactaatatgcatcttgatctcttatggccacattggggtcaccatcctcaaggctccatctactaagggcatcttcaaagctttgtccacctgtggctctcacctctctgtggtgtctctgtattatggcacaattattggactgtattttctcccctcatccagtgcctccagtgacaaggacgtaattgcctctgtgatgtacacggtgatcaccccattgctgaatcccttcatttatagcctaaggaacagggacataaagggagccctggagagactcttcaacagggcaacagtcttatctcaatga

SEQ ID NO: 5 - protein, 324 aa, Homo sapiens
MGMSNLTRLSEFILLGLSSRSEDQRPLFALFLIIYLVTLMGNLLIILAIHSDPRLQNPMYFFLSILSFADICYTTVIVPKMLVNFLSEKKTISYAECLAQMYFFLVFGNIDSYLLAAMAINRCVAICNPFHYVTVMNRRCCVLLLAFPITFSYFHSLLHVLLVNRLTFCTSNVIHHFFCDVNPVLKLSCSSTFVNEIVAMTEGLASVMAPFVCIIISYLRILIAVLKIPSAAGKHKAFSTCSSHLTVVILFYGSISYVYLQPLSSYTVKDRIATINYTVLTSVLNPFIYSLRNKDMKRGLQKLINKIKSQMSRFSTKTNKICGP

SEQ ID NO: 6 - DNA, 975 bp, Homo sapiens
  source 1..975: mol_type = genomic DNA
atgggaatgtccaacctgacaagactctctgaatttattctcttgggactctcctctcggtctgaagaccagaggccactctttgccctctttcttatcatatacctggtcactttgatgggaaatctgctcatcatcttggctatccactctgatcctcgacttcaaaaccctatgtattttttcctaagcatcttgtcctttgctgatatttgctacacaacagtcatagtcccaaagatgctcgtgaacttcttatcagagaaaaagaccatttcctatgctgaatgtctggcacagatgtatttcttcctggtttttggaaacatagatagttatctcctggcggctatggccatcaaccgctgtgtagccatttgtaacccattccattatgtcactgttatgaaccgcagatgctgtgtgttgctactagcattccccatcactttctcctatttccactctctcctacatgtcctcctggtgaatcggctcaccttttgtacatcaaatgttatccatcattttttttgtgatgtcaaccctgtgctgaaactgtcctgctcctccacctttgtcaatgaaattgtggccatgacagaagggctggcctctgtgatggctccatttgtctgtatcatcatctcttatctaagaattctcatcgctgttctcaagattccctcagcagctggaaaacacaaagccttctccacctgcagctcccatctcactgtggtgattctgttttatgggagtattagctatgtctatttgcagcctttgtccagctatactgtcaaggaccgaatagcaacaatcaactacactgtgttgacatcagtgttgaacccatttatctacagtttaagaaacaaagacatgaaacggggcttacagaaattgataaacaagattaagtctcaaatgagtaggttctctacaaagaccaataaaatctgtggaccctga

SEQ ID NO: 7 - protein, 314 aa, Homo sapiens
MDNSNWTSVSHFVLLGISTHPEEQIPLFLVFSLMYAINISGNLAIITLILSAPRLHIPMYIFLSNLALTDICFTSTTVPKMLQIIFSPTKVISYTGCLAQTYFFICFAVMENFILAVMAYDRYIAICHPFHYTMILTRMLCVKMVVMCHALSHLHAMLHTFLIGQLIFCADNRIPHFFCDLYALMKISCTSTYLNTLMIHTEGAVVISGALAFITASYACIILVVLRIPSAKGRWKTFSTCGSHLTVVAIFYGTLSWVYFRPLSSYSVTKGRIITVVYTVVTPMLNPFIYSLRNGDVKGGFMKWMSRMQTFFFR

SEQ ID NO: 8 - DNA, 945 bp, Homo sapiens
  source 1..945: mol_type = genomic DNA
atggacaacagcaactggaccagtgtgtcccattttgttctcttgggcatttccacccacccagaagagcaaatcccactcttccttgttttctcactcatgtacgcaatcaatatttctggcaacttggccatcatcacactgattctctctgctccacgcctccacatccccatgtacatcttcctcagtaacttggccttgacagacatctgcttcacctccaccacggtccccaagatgctgcagattattttctcccctacaaaggtaatttcctacacaggctgtttagcccaaacttatttcttcatttgcttcgccgtcatggaaaacttcatcctggctgtgatggcctatgacaggtacattgccatctgccaccctttccactacactatgatcctgactagaatgctgtgtgtgaagatggtggtcatgtgccatgctctctcccaccttcatgccatgctgcatacctttctcataggccaactaatcttctgtgcagataacagaatcccccacttcttctgtgacctctacgctctgatgaagatctcctgcaccagcacctacctcaacacccttatgattcacacagaaggtgctgttgtaatcagtggagctctggccttcattactgcctcctatgcctgcatcatcctggtggtcctccggatcccctcagccaagggcaggtggaaaaccttttctacctgcggctcccacctcactgtggtggccatattctatggcaccctcagttgggtctacttccggcccctttccagctattcagtgaccaagggtcgcattataacagtcgtgtacacagtggtgactcccatgctgaaccccttcatctacagcctgaggaatggggatgtcaagggaggcttcatgaaatggatgagcagaatgcagacttttttctttagataa

SEQ ID NO: 9 - protein, 316 aa, Homo sapiens
MELRNSTLGSGFILVGILNDSGSPELLYATFTILYMLALTSNGLLLLAITIEARLHMPMYLLLGQLSLMDLLFTSVVTPKALADFLRRENTISFGGCALQMFLALTMGSAEDLLLAFMAYDRYVAICHPLKYMTLMSPRVCWIMVATSWILASLIAIGHTMYTMHLPFCVSWEIRHLLCEIPPLLKLACADTSRYELIIYVTGVTFLLLPISAIVASYTLVLFTVLRMPSNEGRKKALVTCSSHLIVVGMFYGAATFMYVLPSSFHSPKQDNIISVFYTIVTPALNPLIYSLRNKEVMRALRRVLGKYILLAHSTL

SEQ ID NO: 10 - DNA, 4224 bp, Homo sapiens
  source 1..4224: mol_type = genomic DNA
atgaaacacagcatgaggggaatatatgaaatctgaggacctggacttaagaactcagctagaaccattccaaagcagtaaggactttctcgcaaggacagtgaatgagagaactttggaagagattcagctcctcacttctgcaggatcctgccctaggttctcagctgcctgtctgcatctatctctacttccagagaggacttgcagtgccagtgactggaggagcctttgatctctgatcctagagacagccatacacacacctacattcatcctggccagaagaagcaacttttctttctttggcttcttgaacctctttacctggaagctatgccccttcccatatgagcagagcagcacttgaagagtccaggtgatcaagcagcccaagaacccagagattctcctactgaggaaagtaatcaacaacaatgagactggtttaagggagaagacagcaaagaggttttttataaataaaattttaaaaactgaattggaattccaactacattttaagcagaaaaatagctagatcttaaaccaatccccagtatatttgttcattgtgaagaaagagatgctttcatctatctacaaagatgtttgcttgggcttattcttcaggataccttatttgaagggatttctaaatgattactattaacaacatgtcaaaatacctgataccagtttagctgatggtttatttcaataaaagcattcagaaatcagaggcatactaagaaatggaaaatatatcatattatgggattcctattggccaatgcacagtgacctagaacaatccaaagctagagtgaacacctttagaaaagcttccactgataatatatttggttctaggcaactaaaaaaacacatcatggagctccggaactccaccttgggaagcggcttcatcttggtggggattctgaatgacagtgggtctcctgaactgctctatgctacatttacaatcctatacatgttggcactgaccagcaatggtctgctgctcctggccatcaccatagaagcccggctccacatgcccatgtacctcctgcttgggcagctctctctcatggacctcctgttcacatctgttgtcactcccaaggcccttgcggactttctgcgcagagaaaacactatctcctttggaggctgtgcacttcagatgttcctggcactgacaatgggtagcgctgaggacctcctactggccttcatggcctatgacaggtatgtggccatttgtcatcctctgaaatacatgaccctcatgagcccaagagtctgctggatcatggtggccacatcctggatcctggcatccctgattgctataggacataccatgtacactatgcacctccctttctgtgtgtcctgggaaatcaggcatctgctctgtgagatcccacccttgctgaagttggcctgtgctgatacctccaggtatgagcttataatatacgtgacaggtgtgactttcctcttgctccccatttctgccattgtggcctcctacacactagtcctattcactgtgcttcgtatgccatcaaatgaggggaggaagaaagcccttgtcacctgctcttcccacctgattgtggtcgggatgttctatggagctgccacattcatgtatgtcttgcccagttccttccacagccccaaacaagacaacatcatctctgttttctacacaattgtcactccagccctgaatccactcatctacagcctgaggaataaggaggtcatgcgggccttgaggagggtcctgggaaaatacatactgctggcacattccacgctctagggaaggatcatggcttgcctctcaccattcctcctccagataaaattctctactcactcattcctgctccaaataaaattctctactcactcattcttcaatactgtactctgagatgatagatacattttttaaattttacttttattttcaactgtgaatctggtgtgtacaatgatcttgagttaaactcatttggcacacatcatcctgactttggaaacccatgttataaaaataaccagatgggggaaaagccttgaatgggtgttgtagactcactctgtggtaggcatcgtcctgacacattaccaagtagacatggtaggggattagctttccattttaacatcacatagcagtgagaagcagcagtagttttggactgaccaaattccaatagcctctcagcatcagaggaggactactccctttgaagcagattcttaaactactaatagagggaagaattttcagaagatatattagacttcctgtttattaagtagatgattatttgagcagttagtggaaatgaataagagttatgtgactgcatttgtactctagacctaagggaccttcacattggttacttgagtaatgataaataattacattgaaagagtgtggccaggcgcagtagctcacgcctgtaatccaagcactttgggaggctgaggtgggcagatcacctgaggtaaggagttcaagaccagcctggccaacgtgtggaacccgggtctctactaaaaatacaaaaatgagcggggcatagttgtgggtgcctgtaatcccagctactcgggaggctgaggcaggagaattgcttgaccctgggaggcggaggtcgcagtgagctgagatcgcgccactgcactccagcctgagcaacagagagagactccgtttcaataaataaataaataaatagaaagaaagaatggaaaaggataaagaaagatatttctacttggaaattttgacagtctttttgaaaatgggcatttttacattgcctaaaattaaaactaaaataaagaatgaataaaatagaattgaaagtaccagttttaaagtgagtacaataacaatgagagaaatttgttgggaatattgtctaagttttttaaaatgtggacttgcagatatgtgaatgtgtttttaaacattaaaacaattgaaagaggttatattcttgaggtgagtgtatgtacatgtgtgtttccatagaaaaggaagtataaaatttataatttgctttctagctagcctaaataagccttattttttttattctactcatcagtgtgggaagatttatagcaaaatttgggaacttaaatttgttgaatgcagaattcccaaaatatcttggcttacataggtgttattattcttgagaatagtgctttgtcaaaacacaataattcttgttttctttatagaaataatattacattatttaatctataaagctaaaccataaaaaataaaagataaagctaatctataaaattaatccaactttgttataaaaactgagaaaattaaaaaaattagaataaatcaaaaatcacttataaatttttcttttttgttgcacatatatgcattttactgaatttaaatgtccatgatgatgaaggtcctgtgttttttccatttgaaatttggtgaatcttcttccttgctatgagggaatattttagagtagtgatgagaaagcagaccagagtgcccgcatttgaatccctctcttcctttggtcatctatgcatctttgaacgctaaattaaactctttgtaccttagtctgctgatgttaaaagtggtgatatttactggcctgctcttgtgatattgttgggtagattaaataagataattaatatcaggtactgagaccacagcttagtaaactgtgcttaaaacattttggctgctatatggataaatagtaaaaagcctttttaaaaaatcttattttattatttccttttagaaatgaaacatttgtttattatttttattgacaaaatatgtatgtcttaatgtagtaaaacatgatgttttgaaatatgtatactttatggaattgctaaatcaagccaactaacatatgctttactccacatacttatcttttttgcattaggaaaagttaaaatctactttctcagcaattttcaagcacacaatataatctgattaactataattgccatgatctacaataaatctcttgaacttattcctcctgtctaactgaagttttgtgtcctttcaccagcatctcccaaatccccctacctcaacatttggtaaccaccattttactctctgtttctgtaaattcagcttacaggttctgcatataa

SEQ ID NO: 11 - protein, 312 aa, Homo sapiens
MDGVNDSSLQGFVLMGISDHPQLEMIFFIAILFSYLLTLLGNSTIILLSRLEARLHTPMYFFLSNLSSLDLAFATSSVPQMLINLWGPGKTISYGGCITQLYVFLWLGATECILLVVMAFDRYVAVCRPLRYTAIMNPQLCWLLAVIACLGGLGNSVIQSTFTLQLPLCGHRRVEGFLCEVPAMIKLACGDTSLNQAVLNGVCTFFTAVPLSIIVISYCLIAQAVLKIRSAEGRRKAFNTCLSHLLVVFLFYGSASYGYLLPAKNSKQDQGKFISLFYSLVTPMVNPLIYTLRNMEVKGALRRLLGKGREVG

SEQ ID NO: 12 - DNA, 1418 bp, Homo sapiens
  source 1..1418: mol_type = genomic DNA
ttccagcagcttgcgctaaatgaattcatcaagtgactgaagacaaccagtgatggacggggtgaatgatagctccttgcagggctttgttctgatgggcatatcagaccatccccagctggagatgatcttttttatagccatcctcttctcctatttgctgaccctacttgggaactcaaccatcatcttgctttcccgcctggaggcccggctccatacacccatgtacttcttcctcagcaacctctcctccttggaccttgctttcgctactagttcagtcccccaaatgctgatcaatttatggggaccaggcaagaccatcagctatggtggctgcataacccagctctatgtcttcctttggctgggggccaccgagtgcatcctgctggtggtgatggcatttgaccgctacgtggcagtgtgccggcccctccgctacaccgccatcatgaacccccagctctgctggctgctggctgtgattgcctgcctgggtggcttgggcaactctgtgatccagtcaacattcactctgcagctcccattgtgtgggcaccggagggtggagggattcctctgcgaggtgcctgccatgatcaaactggcctgtggcgacacaagtctcaaccaggctgtgctcaatggtgtctgcaccttcttcactgcagtcccactaagcatcatcgtgatctcctactgcctcattgctcaggcagtgctgaaaatccgctctgcagaggggaggcgaaaggcgttcaatacgtgcctctcccatctgctggtggtgttcctcttctatggctcagccagctatgggtatctgcttccggccaagaacagcaaacaggaccagggcaagttcatttccctgttctactcgttggtcacacccatggtgaatcccctcatctacacgctgcggaacatggaagtgaagggcgcactgaggaggttgctggggaaaggaagagaagttggctgagagaacactccttcgttatttattgcgtcttcatctctacatgcgtttctcattaactctctctggccaggtgaacatgaggaatactaattccggtaaaaccaaggcatgttcctgacagccctaagctgacagccctaagctgttgggaacatggttagtgttattcgtaatgttctacacttatttaccaaaaatcctactgtggactaccgatagcaggggagacatgttgttgagggctcagaggttattgacctgtgacagacctgtctcactgtctctgtctctgttgcccacatgttatttaatatgccttatatttctgcagaattctgtacttttctaagcaaagcacctccacttgcagtatcttacataatgttactataattccataaaatcaagccatgtattattaacttca

SEQ ID NO: 13 - protein, 321 aa, Homo sapiens
MLTLNKTDLIPASFILNGVPGLEDTQLWISFPFCSMYVVAMVGNCGLLYLIHYEDALHKPMYYFLAMLSFTDLVMCSSTIPKALCIFWFHLKDIGFDECLVQMFFTHTFTGMESGVLMLMALDRYVAICYPLRYSTILTNPVIAKVGTATFLRGVLLIIPFTFLTKLLPYCRGNILPHTYCDHMSVAKLSCGNVKVNAIYGLMVALLIWGFDILCITNSYTMILRAVVSLSSADARQKAFNTCTAHICAIVFSYTPAFFSFFSHRFGEHIIPPSCHIIVANIYLLLPPTMNPIVYGVKTKQIRDCVIRILSGSKDTKSYSM

SEQ ID NO: 14 - DNA, 1329 bp, Homo sapiens
  source 1..1329: mol_type = genomic DNA
tcagtcagtcattaagaataaaaattaaaaagtcaacatctcatctctcagatcatcagtgaggaaagcccagacaaattttgagctatttcataacctaccagacttatcatgctaacactgaataaaacagacctaataccagcttcatttattctgaatggagtcccaggactggaagacacacaactctggatttccttcccattctgctctatgtatgttgtggctatggtagggaattgtggactcctctacctcattcactatgaggatgccctgcacaaacccatgtactacttcttggccatgctttcctttactgaccttgttatgtgctctagtacaatccctaaagccctctgcatcttctggtttcatctcaaggacattggatttgatgaatgccttgtccagatgttcttcacccacaccttcacagggatggagtctggggtgcttatgcttatggccctggatcgctatgtggccatctgctaccccttacgctattcaactatcctcaccaatcctgtaattgcaaaggttgggactgccaccttcctgagaggggtattactcattattccctttactttcctcaccaagctcctgccctactgcagaggcaatatacttccccatacctactgtgaccacatgtctgtagccaaattgtcctgtggtaatgtcaaggtcaatgccatctatggtctgatggttgccctcctgatttggggctttgacatactgtgtatcaccaactcctataccatgattctccgggcagtggtcagcctctcctcagcagatgctcggcagaaggcctttaatacctgcactgcccacatttgtgccattgttttctcctatactccagctttcttctccttcttttcccaccgctttggggaacacataatccccccttcttgccacatcattgtagccaatatttatctgctcctaccacccactatgaaccctattgtctatggggtgaaaaccaaacagatacgagactgtgtcataaggatcctttcaggttctaaggataccaaatcctacagcatgtgaatgaacacttgccaggagtgagaagagaaggaaagaattacttctatttgcctcttatgcaggagttcataaaatctttctggaagcactgtattgatcacaaaatggagtttgttaactggtgcattctcaataagtaccttgggaatctcaacatcattggaaggcccaccaacatttctataaattttttaccttctcactcatgtgaaggaccagtctaataattaaaccatattttattcgacaaaa

SEQ ID NO: 15 - DNA, 21 bp, synthetic construct
  misc_feature 1..21: note = COL1A1 forward primer
  source 1..21: mol_type = other DNA
acatgttcagctttgtggacc

SEQ ID NO: 16 - DNA, 20 bp, synthetic construct
  misc_feature 1..20: note = COL1A1 reverse primer
  source 1..20: mol_type = other DNA
tgtacgcaggtgattggtgg

SEQ ID NO: 17 - DNA, 23 bp, synthetic construct
  misc_feature 1..23: note = MMP1 forward primer
  source 1..23: mol_type = other DNA
aggggagatcatcgggacaactc

SEQ ID NO: 18 - DNA, 22 bp, synthetic construct
  misc_feature 1..22: note = MMP1 reverse primer
  source 1..22: mol_type = other DNA
gagagtccaagagaatggccga

SEQ ID NO: 19 - DNA, 19 bp, synthetic construct
  misc_feature 1..19: note = p16 forward primer
  source 1..19: mol_type = other DNA
tgcccaacgcaccgaatag

SEQ ID NO: 20 - DNA, 20 bp, synthetic construct
  misc_feature 1..20: note = p16 reverse primer
  source 1..20: mol_type = other DNA
cccctgcaaacttcgtcctc

SEQ ID NO: 21 - DNA, 19 bp, synthetic construct
  misc_feature 1..19: note = GAPDH forward primer
  source 1..19: mol_type = other DNA
aaatcaagtggggcgatgc

SEQ ID NO: 22 - DNA, 20 bp, synthetic construct
  misc_feature 1..20: note = GAPDH reverse primer
  source 1..20: mol_type = other DNA
agggggcagagatgatgacc

SEQ ID NO: 23 - DNA, 20 bp, synthetic construct
  misc_feature 1..20: note = OR1J2 forward primer
  source 1..20: mol_type = other DNA
tgtcaccctctccactacac

SEQ ID NO: 24 - DNA, 22 bp, synthetic construct
  misc_feature 1..22: note = OR1J2 reverse primer
  source 1..22: mol_type = other DNA
ctggcacaagacagaatccaag

SEQ ID NO: 25 - DNA, 21 bp, synthetic construct
  misc_feature 1..21: note = OR1J4 forward primer
  source 1..21: mol_type = other DNA
gtcaccatcctcaaggctcca

SEQ ID NO: 26 - DNA, 21 bp, synthetic construct
  misc_feature 1..21: note = OR1J4 reverse primer
  source 1..21: mol_type = other DNA
accgtgtacatcacagaggca

SEQ ID NO: 27 - DNA, 21 bp, synthetic construct
  misc_feature 1..21: note = OR1L3 forward primer
  source 1..21: mol_type = other DNA
ttctcttgggactctcctctc

SEQ ID NO: 28 - DNA, 22 bp, synthetic construct
  misc_feature 1..22: note = OR1L3 reverse primer
  source 1..22: mol_type = other DNA
cacgagcatctttgggactatg

SEQ ID NO: 29 - DNA, 20 bp, synthetic construct
  misc_feature 1..20: note = OR1Q1 forward primer
  source 1..20: mol_type = other DNA
ttctctctgctccacgcctc

SEQ ID NO: 30 - DNA, 20 bp, synthetic construct
  misc_feature 1..20: note = OR1Q1 reverse primer
  source 1..20: mol_type = other DNA
tttccatgacggcgaagcaa

SEQ ID NO: 31 - DNA, 22 bp, synthetic construct
  misc_feature 1..22: note = OR2AG2 forward primer
  source 1..22: mol_type = other DNA
tctacagcctgaggaataagga

SEQ ID NO: 32 - DNA, 20 bp, synthetic construct
  misc_feature 1..20: note = OR2AG2 reverse primer
  source 1..20: mol_type = other DNA
ggaggaatggtgagaggcaa

SEQ ID NO: 33 - DNA, 21 bp, synthetic construct
  misc_feature 1..21: note = OR2C1 forward primer
  source 1..21: mol_type = other DNA
tcaatacgtgcctctcccatc

SEQ ID NO: 34 - DNA, 21 bp, synthetic construct
  misc_feature 1..21: note = OR2C1 reverse primer
  source 1..21: mol_type = other DNA
ttcttggccggaagcagatac

SEQ ID NO: 35 - DNA, 20 bp, synthetic construct
  misc_feature 1..20: note = OR52N4 forward primer
  source 1..20: mol_type = other DNA
ccaagctcctgccctactgc

SEQ ID NO: 36 - DNA, 20 bp, synthetic construct
  misc_feature 1..20: note = OR52N4 reverse primer
  source 1..20: mol_type = other DNA
aaggccttctgccgagcatc

SEQ ID NO: 37 - DNA, 19 bp, synthetic construct
  misc_feature 1..19: note = GAPDH forward primer
  source 1..19: mol_type = other DNA
aaatcaagtggggcgatgc

SEQ ID NO: 38 - DNA, 20 bp, synthetic construct
  misc_feature 1..20: note = GAPDH reverse primer
  source 1..20: mol_type = other DNA
agggggcagagatgatgacc